(19) United States
(12) Patent Application Publication
(10) Pub. No.: US 2020/0354411 A1
(43) Pub. Date: Nov. 12, 2020

(54) Chimeric Molecule for Targeting c-Myc in Cells

(71) Applicant: AGENCY FOR SCIENCE, TECHNOLOGY AND RESEARCH, Singapore (SG)

(72) Inventors: Frederic Bard, Singapore (SG); Alexandre Chaumet, Singapore (SG); Trinda Anne Ting, Singapore (SG)

(21) Appl. No.: 16/760,060

(22) PCT Filed: Nov. 29, 2018
(86) PCT No.: PCT/SG2018/050584

§ 371 Date: Apr. 29, 2020

(30) Foreign Application Priority Data
Nov. 29, 2017 (SG) 10201709898X

(51) Int. Cl. C07K 14/21; C07K 9/00; A61P 35/00

(52) U.S. Cl. CPC C07K 14/21; C07K 2319/55; A61P 35/00; C07K 9/00

(57) ABSTRACT

Disclosed herein are chimeric fusion proteins comprising a c-Myc inhibitor fused to genetically modified Pseudomonas Aeroginosa Exotoxin A (‘tPE’), said fusion proteins being capable of penetrating a nucleus of a cell and inhibiting c-Myc activity within the nucleus. Also disclosed herein are pharmaceutical compositions comprising chimeric fusion proteins, methods of delivering, methods of preparing, methods of treating and uses of chimeric fusion proteins.

This application claims the benefit of Singaporean Provisional Application No. 10201709898X filed on 29 Nov. 2017, the content of which is incorporated herein by reference in its entirety.

### TECHNICAL FIELD

This invention relates to the field of cancer and, more particularly, to compositions and methods for the treatment of cancer using a chimeric fusion protein comprising genetically modified Pseudomonas Aeroginosa Exotoxin A fused to a c-Myc inhibitor that can target and penetrate a nucleus of a cell, and inhibit the activity of the transcription factor c-Myc within the nucleus.

### BACKGROUND

c-myc is a regulator gene that codes for the transcription factor c-Myc. A mutated version of c-Myc is found in many cancers, which causes c-Myc to be constitutively expressed and display oncogenic activity. This leads to the unregulated expression of many genes, some of which are involved in cell proliferation, and results in the formation of cancer.

The frequency of genetic alterations of c-myc in human cancers has allowed an estimation that approximately 70,000 U.S. cancer deaths per year are associated with changes in the c-myc gene or its expression. Given that c-myc may contribute to one-seventh of U.S. cancer deaths, recent efforts have been directed toward inhibition of the c-Myc protein in cancer biology with the hope that therapeutic insights will emerge.

Therefore, there is an unmet need for a method of detecting and or targeting c-Myc in cells.

### SUMMARY

In one aspect, the present invention refers to a chimeric fusion protein comprising a c-Myc inhibitor fused to genetically modified Pseudomonas Aeroginosa Exotoxin A (‘tPE’), said fusion protein being capable of penetrating a nucleus of a cell and inhibiting c-Myc activity within the nucleus.

In another aspect, the present invention refers to a pharmaceutical composition comprising a chimeric fusion protein comprising a c-Myc inhibitor fused to genetically modified Pseudomonas Aeroginosa Exotoxin A (‘tPE’), said fusion protein being capable of penetrating a nucleus of a cell and inhibiting c-Myc activity within the nucleus.

In yet another aspect, the present invention refers to a method of delivering a c-Myc inhibitor to a nucleus of a cell, said method comprising the step of subjecting a chimeric fusion protein comprising said c-Myc inhibitor fused to genetically modified Pseudomonas Aeroginosa Exotoxin A (‘tPE’) to said cell, wherein said fusion protein is capable of penetrating a nucleus of the cell and inhibiting c-Myc activity within the nucleus.

In a further aspect, the present invention refers to a method of delivering a c-Myc inhibitor to a nucleus of a cell, said method comprising the steps of fusing said c-Myc inhibitor with genetically modified Pseudomonas Aeroginosa Exotoxin A (‘tPE’) to create a chimeric fusion protein capable of penetrating a nucleus of the cell and inhibiting c-Myc activity within the nucleus, and subjecting said fusion protein to said cell.

In another aspect, the present invention refers to a method of manufacturing a chimeric fusion protein capable of penetrating a nucleus of a cell, said method comprising the step of fusing a c-Myc inhibitor with genetically modified Pseudomonas Aeroginosa Exotoxin A (‘tPE’) to create the chimeric fusion protein capable of penetrating a nucleus of the cell and inhibiting c-Myc activity within the nucleus.

In yet another aspect, the present invention refers to a method of preventing or treating a c-Myc-dependent cancer in a subject, said method comprising the step of administering to the subject a c-Myc inhibitor fused to genetically modified Pseudomonas Aeroginosa Exotoxin A (‘tPE’), said fusion protein being capable of penetrating a nucleus of a cell of the subject and inhibiting c-Myc activity within the nucleus.

In one aspect, the present invention refers to a chimeric fusion protein for use in preventing or treating a c-Myc-dependent cancer in a subject, wherein said chimeric fusion protein comprises a c-Myc inhibitor fused to genetically modified Pseudomonas Aeroginosa Exotoxin A (‘tPE’), said fusion protein being capable of penetrating a nucleus of a cell of the subject and inhibiting c-Myc activity within the nucleus.

In another aspect, the present invention refers to use of a chimeric fusion protein in the manufacture of a medicament for preventing or treating a c-Myc-dependent cancer in a subject, wherein said chimeric fusion protein comprises a c-Myc inhibitor fused to genetically modified Pseudomonas Aeroginosa Exotoxin A (‘tPE’), said fusion protein being capable of penetrating a nucleus of a cell of the subject and inhibiting c-Myc activity within the nucleus.

### BRIEF DESCRIPTION OF FIGURES

Various embodiments of the invention will be described with reference to the following Figures.

FIG. 1: Schematic of the tPE-Omomyc and tPE-H1 primary structures and Principle of the invention. (A) shows the primary structures of each of tPE-Omomyc and tPE-H1. For the tPE-Omomyc fusion proteins, PE domains Ia and II are genetically fused to Omomyc peptide. For the tPE-H1 fusion proteins, PE domains Ia and II are genetically fused to H1 peptide. (B) shows a schematic of the E-BOX under normal conditions, and under c-Myc conditions. When cells are incubated with tPE-Omomyc, the c-Myc/Max complex can no longer bind to E-Box promoter. In a similar fashion, when cells are incubated with tPE-H1, neither the max peptide nor the c-Myc protein (bound to the tPE-H1 fusion protein) can bind to E-Box promoter. Cells express luciferase upon E-Box promoter to measure c-myc activity and Renilla upon CMV promoter as a control.

FIG. 2: Subcellular location. (A) shows A431 cell fractionation after 1 hour tPE-H1 200 nM treatment shows tPE-H1, c-Myc and Max location in the nucleus of the cells. C: Cytosolic fraction; M: Membrane fraction; N: Nuclear fraction. Antibodies are labelled on the left of each blot. Molecular weight is shown on the right. (B) shows A431 cell fractionation after 1 hour tPE-Omomyc 200 nM treatment shows tPE-Omomyc, in the nuclear fraction. Alpha-tubulin is used as cytosolic fraction marker, Calnexin is used as membrane fraction marker and Max is used as a nuclear fraction marker. C: Cytosolic fraction, M: Membrane fraction, N: Nuclear fraction. Antibodies are labelled on the left of each blot. MW are shown on the right. Results show the presence of tPE-Omomyc in the nuclear fraction 1 hour after incubation.

FIG. 3: Effect of tPE-H1 on c-Myc/Max complex. C-Myc co-immunoprecipitation with Max in absence of c-Myc antibody (−Ab, negative control) or in presence of c-Myc antibody (IP c-Myc). Cells were treated with tPE (ie. PE Domains Ia and II without a H1 peptide) and tPE-H1 to test their effect of c-Myc/Max interaction. Mock sample contains no form of tPE. Antibodies are labelled on the left of each blot. Molecular weight is shown on the right.

FIG. 4: tPE-H1 dose response and EC50 on c-Myc transcriptional activity. tPE-H1 dose response on A431 cells expressing luciferase upon E-Box promoter control at 6 hours. Cells were treated with several tPE-H1 concentrations. Calculated EC₅₀=25 nM.

FIG. 5: tPE-H1 kinetic on c-Myc transcriptional activity. tPE-H1 50 nM kinetic effect on A431 cells expressing luciferase upon E-Box promoter control. Cells were treated and luciferase activity was read at different time points. The peak activity occurs at 6 to 8 hours and remains stable.

FIG. 6: tPE-H1 summary results and c-Myc specificity. A431 cells expressing luciferase upon E-Box promoter control and Renilla under CMV promoter control are treated for 6 hours with 50 nM tPE (negative control; SEQ ID NO: 10), tPE-H1 (SEQ ID NO: 14), which comprises a mutated version of the H1 negative control) or tPE-H1-control (SEQ ID NO: 13), respectively. Results show a decrease of the luciferase when cells are treated with tPE-H1 but not tPE nor tPE-H1-control (black bars). The Renilla luciferase is not affected by the treatment (grey bars). Results show the specificity of tPE-H1 on E-Box luciferase.

FIG. 7: CPP-H1 dose response and EC₅₀ on c-Myc transcriptional activity. Comparative dose response after 6 hours treatment of cell targeting peptides (CPP) fused to H1 and tPE-H1 on A431 cells expressing luciferase upon E-Box promoter control. X axis is shown in Log Cadherin (CAD; LLIILLRRRIRKQAHAHSK; SEQ ID NO: 2) EC₅₀=75 μM, Antenapedia (Int; RQIKIWFQNRRMKWKK SEQ ID NO: 3) EC₅₀=200 μM and TAT (GRKKRRQRRRPPQ; SEQ ID NO: 4) EC₅₀=500 μM.

FIG. 8: tPE-H1 effect on A431 cell proliferation. A431 cells were treated with 50, 100, 200 and 400 nM tPE-H1 over 2 weeks. Bright field acquisition was made every 4 hours and analysed. Results show a slower cell proliferation at 50 nM and 100 nM tPE-H1 and no proliferation above 200 nM.

FIG. 9: tPE-H1 effect on Hepatocarcinoma HepG2 cell proliferation. HepG2 cells were treated with 10 nM, 25 nM, 50 nM and 100 nM tPE-H1 over 2 weeks. Bright field acquisition was made every 4 hours and analysed. Results show a slower cell proliferation at 10 nM and 25 nM tPE-H1, and no proliferation above 50 nM.

FIG. 10: Hepatocarcinoma HepG2 mortality under tPE-H1 treatment. HepG2 were treated with (black bars) or without (grey bars) 100 nM tPE-H1 for 24 hours in presence of cells death marker DRAQ7. Positive DRAQ7 cells were counted and compared in each condition. Results show an increase of the number of dead cells 24 hours after treatment with tPE-H1 100 nM.

FIG. 11: tPE-H1 effect on c-Myc biomarker. A431 cells were treated with tPE H1 100 nM overnight before RNA extraction. Transcripts mRNA amount regulated by c Myc were quantified by RT-PCRQ and compared with or without tPE-H1 treatment. Housekeeping mRNAs (HPRT1, GAPDH) which expression are not regulated by c-Myc are analysed the same manner. Y axis shows the mRNA log 2 (fold change). Upregulated genes appear with negative log 2 (fold change) compare to housekeeping gene. Downregulated genes appear with positive log 2 (fold change) compare to housekeeping gene.

FIG. 12: tPE-Omomyc dose response and EC₅₀ on c-Myc transcriptional activity. tPE-Omomyc dose response (depicted as a line graph) on A431 cells expressing luciferase upon E-BOX promoter control at 6 hours. Cells were treated with several tPE-Omomyc concentrations, as shown on the X-axis of FIG. 12. Results show a calculated EC₅₀=5 nM after 6 hours incubation, which is 5 times lower than results obtained with tPE-H1.

FIG. 13: tPE-Omomyc kinetic on c-Myc transcriptional activity. FIG. 13 shows a line graph depicting the kinetic effect of tPE-Omomyc 10 nM on A431 cells expressing luciferase upon E-Box promoter control. Cells were treated and luciferase activity was read at different time points. Results show that the peak activity occurs at 16 hours after incubation and remains stable.

FIG. 14: tPE-Omomyc effect on Hepatocarcinoma HepG2 cell proliferation. HepG2 cells were treated with 1 nM, 2.5 nM, 5 nM and 10 nM tPE-Omomyc for 2 weeks. Bright field acquisition was performed every 4 hours and analysed. Results show a slower cell proliferation at 2.5 nM and 5 nM tPE-Omomyc and no proliferation above 10 nM.

FIG. 15: Hepatocarcinoma HepG2 cell mortality upon tPE-Omomyc treatment. HepG2 were treated with (black bars) or without (white bars) 10 nM tPE-Omomyc for 24 hours in presence of cells death marker DRAQ7. Positive DRAQ7 cells were counted and compared for each condition. Results show an increase of the number of dead cells 24 hours after treatment with tPE-Omomyc at a concentration of 10 nM.

LISTING OF SEQUENCES

| SEQ | | |
|---|---|---|
| ID | | |
| NO: | Description | Sequence |
| 1 | H1-control (H1-neg; | NELKRSFFALRDQI |
| | original unmutated H1 | |
| | sequence) | |
| 2 | Cadherin (CAD) | LLIILLRRRIRKQAHAHSK |
| 3 | Antenapedia (Int) | RQIKIWFQNRRMKWKK |
| 4 | TAT | GRKKRRQRRRPPQ |
| 5 | Mutated H1 (S6A, F8A; | NELKRAFAALRDQI |
| | also referred to as tPE- | |
| | H1) | |
| 6 | Omomyc | TEENVKRRTHNVLERQRRNELKRSFFALRD |
| | | QIPELENNEKAPKVVILKKATAYILSVQAETQ |
| | | KLISEIDLLRKQNEQLKHKLEQLRNSCA |
| 7 | Int-H1 | RQIKIWFQNRRMKWKK NELKRAFAALRDQI |
| 8 | CAD-H1 | LLIILLRRRIRKQAHAHSK NELKRAFAALRDQI |
| 9 | Tat-H1 | GRKKRRQRRRPPQ NELKRAFAALRDQI |
| 10 | tPE (comprising both PE | MEEAFDLWNECAKACVLDLKDGVRSSRMSVD |
| | domains Ia and II) | PAIADTNGQGVLHYSMVLEGGNDALKLAIDNAL |
| | | SITSDGLTIRLEGGVEPNKPVRYSYTRQARGSW |
| | | SLNWLVPIGHEKPSNIKVFIHELNAGNQLSHMS |
| | | PIYTIEMGDELLAKLARDATFFVRAHESNEMQP |
| | | TLAISHAGVSVVMAQAQPRREKRWSEWASGK |
| | | VLCLLDPLDGVYNYLAQQRCNLDDTWEGKIYR |
| | | VLAGNPAKHDLDIKPTVISHRLHFPEGGSLAALT |
| | | AHQACHLPLETFTRHRQPRGWEQLEQCGYPV |
| | | QRLVALYLAARLSWNQVDQVIRNALASPGSGG |
| | | DLGEAIREQPEQARLALTLAAAESERFVRQGTG |
| | | NDEAGAAS |
| 11 | tPE comprising only the | EEAFDLWNECAKACVLDLKDGVRSSRMSVDPA |
| | PE Domain Ia | IADTNGQGVLHYSMVLEGGNDALKLAIDNALSIT |
| | | SDGLTIRLEGGVEPNKPVRYSYTRQARGSWSL |
| | | NWLVPIGHEKPSNIKVFIHELNAGNQLSHMSPIY |
| | | TIEMGDELLAKLARDATFFVRAHESNEMQPTLAI |
| | | SHAGVSVVMAQAQPRREKRWSEWASGKVLCL |
| | | LDPLDGVYNYLAQQRCNLDDTWEGKIYRVLAG |
| | | NPAKHDLDIKPTVISHRLHFPE |
| 12 | tPE comprising only the | GGSLAALTAHQACHLPLETFTRHRQPRGWEQL |
| | PE Domain II | EQCGYPVQRLVALYLAARLSWNQVDQVIRNAL |
| | | ASPGSGGDLGEAIREQPEQARLALTLAAAESER |
| | | FVRQGTGNDEAGAAS |
| 13 | tPE-H1-control (tPE-H1- | MEEAFDLWNECAKACVLDLKDGVRSSRMSVD |
| | neg; H1 portion is | PAIADTNGQGVLHYSMVLEGGNDALKLAIDNAL |
| | underlined) | SITSDGLTIRLEGGVEPNKPVRYSYTRQARGSW |
| | | SLNWLVPIGHEKPSNIKVFIHELNAGNQLSHMS |
| | | PIYTIEMGDELLAKLARDATFFVRAHESNEMQP |
| | | TLAISHAGVSVVMAQAQPRREKRWSEWASGK |
| | | VLCLLDPLDGVYNYLAQQRCNLDDTWEGKIYR |
| | | VLAGNPAKHDLDIKPTVISHRLHFPEGGSLAALT |
| | | AHQACHLPLETFTRHRQPRGWEQLEQCGYPV |
| | | QRLVALYLAARLSWNQVDQVIRNALASPGSGG |
| | | DLGEAIREQPEQARLALTLAAAESERFVRQGTG |
| | | NDEAGAASNELKRSFFALRDQI |
| 14 | tPE-H1 (mutated H1 | MEEAFDLWNECAKACVLDLKDGVRSSRMSVD |
| | sequence underlined) | PAIADTNGQGVLHYSMVLEGGNDALKLAIDNAL |
| | | SITSDGLTIRLEGGVEPNKPVRYSYTRQARGSW |
| | | SLNWLVPIGHEKPSNIKVFIHELNAGNQLSHMS |
| | | PIYTIEMGDELLAKLARDATFFVRAHESNEMQP |
| | | TLAISHAGVSVVMAQAQPRREKRWSEWASGK |
| | | VLCLLDPLDGVYNYLAQQRCNLDDTWEGKIYR |
| | | VLAGNPAKHDLDIKPTVISHRLHFPEGGSLAALT |
| | | AHQACHLPLETFTRHRQPRGWEQLEQCGYPV |
| | | QRLVALYLAARLSWNQVDQVIRNALASPGSGG |
| | | DLGEAIREQPEQARLALTLAAAESERFVRQGTG |
| | | NDEAGAASNELKRAFAALRDQI |
| 15 | tPE-Omomyc (Omomyc | MEEAFDLWNECAKACVLDLKDGVRSSRMSVD |
| | sequence underlined) | PAIADTNGQGVLHYSMVLEGGNDALKLAIDNAL |
| | | SITSDGLTIRLEGGVEPNKPVRYSYTRQARGSW |
| | | SLNWLVPIGHEKPSNIKVFIHELNAGNQLSHMS |
| | | PIYTIEMGDELLAKLARDATFFVRAHESNEMQP |
| | | TLAISHAGVSVVMAQAQPRREKRWSEWASGK |
| | | VLCLLDPLDGVYNYLAQQRCNLDDTWEGKIYR |
| | | VLAGNPAKHDLDIKPTVISHRLHFPEGGSLAALT |
| | | AHQACHLPLETFTRHRQPRGWEQLEQCGYPV |
| | | QRLVALYLAARLSWNQVDQVIRNALASPGSGG |
| | | DLGEAIREQPEQARLALTLAAAESERFVRQGTG |
| | | NDEAGAASTEENVKRRTHNVLERQRRNELKRS |
| | | FFALRDQIPELENNEKAPKVVILKKATAYILSVQA |
| | | ETQKLISEIDLLRKQNEQLKHKLEQLRNSCA |
| 16 | Sec61B siRNA sequence | GCAAGUACACUCGUUCGUA |
| 17 | SUN2 siRNA sequence | CCUAUGGGCUGCAGACAUU |

### DETAILED DESCRIPTION

The inventors have developed a genetically modified version of Pseudomonas Aeroginosa Exotoxin A (‘tPE’) that is capable of targeting and penetrating a nucleus of a cell, and can be used to target/deliver a therapeutic/biologically active peptide or protein to the nucleus. More particularly, the inventors have developed a chimeric fusion protein comprising a c-Myc inhibitor fused to the tPE that can enter a cell's nucleus. The inventors have discovered that the fusion protein can be used to treat c-Myc-dependent cancers.

According to a first embodiment of the present invention, there is provided a chimeric fusion protein comprising a c-Myc inhibitor fused to genetically modified Pseudomonas Aeroginosa Exotoxin A (‘tPE’), said fusion protein being capable of penetrating a nucleus of a cell and inhibiting c-Myc activity within the nucleus.

According to a second embodiment of the present invention, there is provided a pharmaceutical composition comprising a chimeric fusion protein comprising a c-Myc inhibitor fused to genetically modified Pseudomonas Aeroginosa Exotoxin A (‘tPE’), said fusion protein being capable of penetrating a nucleus of a cell and inhibiting c-Myc activity within the nucleus.

According to a third embodiment of the present invention, there is provided a method of delivering a c-Myc inhibitor to a nucleus of a cell, said method comprising the step of subjecting a chimeric fusion protein comprising said c-Myc inhibitor fused to genetically modified Pseudomonas Aeroginosa Exotoxin A (‘tPE’) to said cell, wherein said fusion protein is capable of penetrating a nucleus of the cell and inhibiting c-Myc activity within the nucleus.

According to a fourth embodiment of the present invention, there is provided a method of delivering a c-Myc inhibitor to a nucleus of a cell, said method comprising the steps of fusing said c-Myc inhibitor with genetically modified Pseudomonas Aeroginosa Exotoxin A (‘tPE’) to create a chimeric fusion protein capable of penetrating a nucleus of the cell and inhibiting c-Myc activity within the nucleus, and subjecting said fusion protein to said cell.

According to a fifth embodiment of the present invention, there is provided a method of manufacturing a chimeric fusion protein capable of penetrating a nucleus of a cell, said method comprising the step of fusing a c-Myc inhibitor with genetically modified Pseudomonas Aeroginosa Exotoxin A (‘tPE’) to create the chimeric fusion protein capable of penetrating a nucleus of the cell and inhibiting c-Myc activity within the nucleus.

According to a sixth embodiment of the present invention, there is provided a method of preventing or treating a c-Myc-dependent cancer in a subject, said method comprising the step of administering to the subject a c-Myc inhibitor fused to genetically modified Pseudomonas Aeroginosa Exotoxin A (‘tPE’), said fusion protein being capable of penetrating a nucleus of a cell of the subject and inhibiting c-Myc activity within the nucleus.

According to a seventh embodiment of the present invention, there is provided a chimeric fusion protein for use in preventing or treating a c-Myc-dependent cancer in a subject, wherein said chimeric fusion protein comprises a c-Myc inhibitor fused to genetically modified Pseudomonas Aeroginosa Exotoxin A (‘tPE’), said fusion protein being capable of penetrating a nucleus of a cell of the subject and inhibiting c-Myc activity within the nucleus.

According to an eighth embodiment of the present invention, there is provided use of a chimeric fusion protein in the preparation of a medicament for preventing or treating a c-Myc-dependent cancer in a subject, wherein said chimeric fusion protein comprises a c-Myc inhibitor fused to genetically modified Pseudomonas Aeroginosa Exotoxin A (‘tPE’), said fusion protein being capable of penetrating a nucleus of a cell of the subject and inhibiting c-Myc activity within the nucleus.

The genetically modified version of Pseudomonas Aeroginosa Exotoxin A (‘tPE’) can be of any suitable form, provided that it is capable of being taken up into the cell via endocytosis, as well as target and penetrate the nucleus of the cell. The tPE can therefore comprise one or more domains that enable it to translocate across membranes of the cell, including the outer cell membrane and nuclear membrane. The tPE can be produced in any suitable way.

In some examples, the tPE comprises Domain Ia (amino acids 1-252 of the mature cleaved protein) or a biologically active fragment thereof for translocating across a nuclear membrane. In other examples, the tPE comprises Domain II (amino acids 253-364 of the mature cleaved protein) or a biologically active fragment thereof for translocating across a nuclear membrane.

In another example, the tPE comprises Domain Ia (amino acids 1-252 of the mature cleaved protein) or a biologically active fragment thereof as well as Domain II (amino acids 253-364 of the mature cleaved protein) or a biologically active fragment thereof. In yet another example, the tPE comprises Domain Ia (amino acids 1-252 of the mature cleaved protein) fused to Domain II (amino acids 253-364 of the mature cleaved protein).

Myc is a family of regulator genes and proto-oncogenes that code for transcription factors, the most well-known example of which is c-myc. Other examples of Myc are 1-myc, and n-myc. In cancer, c-myc is often constitutively (and possibly persistently) expressed, which in turn leads to an increased expression of many other genes, some of which are thought to be involved in cell proliferation. Therefore, without being bound by theory, it is thought that the overall c-myc expression contributes to the formation of cancer. Constitutive up-regulation of Myc genes have also been observed in carcinoma of the cervix, colon, breast, lung and stomach. In the human genome, c-myc is believed to regulate expression of about 15% of all genes through binding on so-called enhancer box sequences (E-Boxes).

As used herein, the term “inhibitor” refers to compounds that are capable of inhibiting or blocking the activity of a specific target. These targets can be, but are not limited to, enzymes, receptors (neurotransmitters being a non-limiting examples), proteins, genes and any other molecules that have a biological function. Various compounds and drugs are not limited to a single effect and can therefore be considered to be inhibitors for a specific target, even if they are structurally different. That is to say, the inhibition of the specific target is the combining characteristic of these compounds.

Thus, in one example, the inhibitors disclosed herein are c-Myc inhibitors. Any suitable type of c-Myc inhibitor can be used in conjunction with the subject matter disclosed herein, provided that it is capable of directly or indirectly inhibiting c-Myc within the nucleus. The c-Myc inhibitor can be produced in any suitable way.

In one example, the c-Myc inhibitor is fused to the C-terminus of the tPE. In yet another example, the c-Myc inhibitor is fused to the C-terminus of Domain II of the tPE.

In some embodiments, the c-Myc inhibitor can be a peptide of any suitable sequence and length. In some embodiments, the c-Myc inhibitor can be a polypeptide of any suitable sequence and length. In some embodiments, the c-Myc inhibitor can comprise more two or more peptides fused to the tPE. The peptides can be the same or different from each other. In some embodiments, the c-Myc inhibitor can comprise more two or more polypeptides fused to the tPE. The polypeptides can be the same or different from each other. In some embodiments, the c-Myc inhibitor can comprise more two or more peptides and/or polypeptides fused to the tPE. The peptides and polypeptides can be the same or different from each other.

In some embodiments, the c-Myc inhibitor directly or indirectly inhibits c-Myc. In some embodiments, the c-Myc inhibitor is capable of disrupting c-Myc dependent pathways in c-Myc-dependent cancers. In some embodiments, the c-Myc inhibitor interferes with specific c-Myc DNA binding. In some embodiments, the c-Myc inhibitor blocks c-Myc/Max dimerization, thereby inhibiting transcription activation by c-Myc.

In yet another example, the c-Myc inhibitor is a H1 peptide derived from the helix 1 (H1) carboxylic region of c-Myc that can interfere with specific c-Myc DNA binding. The H1 peptide can be of any suitable sequence and length, but is preferably H1 (S6A, F8A) having the amino acid sequence NELKRAFAALRDQI (SEQ ID NO.: 5). Other H1 c-Myc-inhibiting peptide sequences of interest include, for example, Omomyc (TEENVKRRTHNVLERQRRNELKRSFFALRDQIPELENNEKAPKVVILKKATAYILSVQAETQKLI SEIDLLRKQNEQLKHKLEQLRNSCA; SEQ ID NO: 6).

As used herein, the terms “peptide”, “protein”, “polypeptide”, and “amino acid sequence” are used interchangeably herein to refer to polymers of amino acid residues of any length. The polymer may be linear or branched, it may comprise modified amino acids or amino acid analogues, and it may be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labelling or bioactive component. The term peptide encompasses two or more naturally occurring or synthetic amino acids linked by a covalent bond (e.g., an amide bond). The amino acid residues are joined together through amide bonds. When the amino acids are alpha-amino acids, either the L-optical isomer or the D-optical isomer can be used, the L-isomers being preferred in nature. The term polypeptide or protein as used herein encompasses any amino acid sequence and includes, but may not be limited to, modified sequences such as glycoproteins. The term polypeptide is specifically intended to cover naturally occurring proteins, as well as those that are recombinantly or synthetically produced. Substantially purified polypeptide as used herein refers to a polypeptide that is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative amino acid substitution tables providing functionally similar amino acids are well known to one of ordinary skill in the art. The following six groups are examples of amino acids that are considered to be conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

A non-conservative amino acid substitution can result from changes in: (a) the structure of the amino acid backbone in the area of the substitution; (b) the charge or hydrophobicity of the amino acid; or (c) the bulk of an amino acid side chain. Substitutions generally expected to produce the greatest changes in protein properties are those in which: (a) a hydrophilic residue is substituted for (or by) a hydrophobic residue; (b) a proline is substituted for (or by) any other residue; (c) a residue having a bulky side chain, e.g., phenylalanine, is substituted for (or by) one not having a side chain, e.g., glycine; or (d) a residue having an electropositive side chain, e.g., lysyl, arginyl, or histadyl, is substituted for (or by) an electronegative residue, e.g., glutamyl or aspartyl.

Variant amino acid sequences may, for example, be 80%, 85%, 90% or even 95%, 98% or 99% identical to the native amino acid sequence. Programs and algorithms for determining percentage identity can be performed according to methods known in the art.

The cell can be an isolated mammalian cell, for example, such as cells that are cultured in vitro (cell culture) or cells that have been obtained from a subject. In one example, the cell is a human cell. In another example, the subject is, but is not limited to, human, canine, porcine, bovine, murine, rodent, feline, primates (including non-human primates) and equine. That is, treatment, exposure, contacting or administration of the chimeric protein to the mammalian cell can be carried out in vitro or ex vivo.

The mammalian cell can be of any suitable type. It can be a human cell, a primate cell, a cell of a laboratory animal (such as a rodent or rabbit, for example), a cell of a farm animal or livestock (such as a horse, sheep, goat or bovine), or a cell of a companion animal (such as a dog or cat).

Likewise, the subject can be a human, primate, laboratory animal, farm animal, livestock or companion animal.

In some embodiments, the c-Myc dependent cancer is a carcinoma or tumour of the cervix, colon, breast, lung or stomach.

In some embodiments, the chimeric fusion protein can comprise a synthetic tag, such as a polyhistidine tag, HQ tag, HN tag, FLAG tag or HAT tag, or multiples thereof, for protein production and purification purposes. In some embodiments, a polyhistidine tag can be fused to an N-terminus of tPE Domain Ia. The polyhistidine tag can be, for example, 5, 6, 7, 8, 9, 10, 11, 12, 13, 14, 15, 16, 17, 18, 19, 20, 21, 22, 23, 24, 25, 26, 27, 28, 29, 30 or more residues in length.

The pharmaceutical composition can comprise a pharmaceutically acceptable carrier or one or more other ingredients.

The chimeric fusion protein or pharmaceutical composition (hereafter “composition”) can be administered to the subject in either a prophylactically effective or a therapeutically effective amount as necessary for the particular situation under consideration. The actual amount of the composition and rate and time-course of administration of the composition, will depend on the nature and severity of the cancer being treated or the prophylaxis required. Prescription of treatment such as decisions on dosage and the like will be within the skill of the medical practitioner or veterinarian responsible for the care of the subject. Typically however, compositions for administration to a subject will include between about 0.01 mg and 100 mg of the compound per kg of body weight. In another example, the composition disclosed herein is to be administered at an amount between about 0.1 and 10 mg/kg of body weight. In yet another examples, the composition or compound is to be administered at an amount of between 0.1 mg/kg and 10 mg/kg, between 0.1 mg/kg and 5 mg/kg, between 1 mg/kg to 2.5 mg/kg, between 2.5 mg/kg to 5 mg/kg, between 5 mg/kg and 10 mg/kg, between 5 mg/kg and 7.5 mg/kg, between 7.5 mg/kg and 10 mg/kg, at least 1 mg/kg, at least 1.5 mg/kg, at least 1.8 mg/kg, at least 2 mg/kg, at least 2.5 mg/kg, at least 2.8 mg/kg, at least 3 mg/kg, at least 3.2 mg/kg, at least 3.5 mg/kg, at least 4 mg/kg, at least 4.5 mg/kg, at least 5 mg/kg, at least 5.5 mg/kg, at least 6 mg/kg, at least 6.5 mg/kg, at least 7 mg/kg, at least 7.5 mg/kg, at least 8 mg/kg, at least 8.5 mg/kg, at least 9 mg/kg, at least 9.5 mg/kg or at least 10 mg/kg.

In one example, the amounts to be administered, as described herein, are to be understood as the dosage regime per day. In another example, the medicament is to be administered to a subject daily, weekly, twice a week (bi-weekly), three times a week, every two weeks, monthly (that is to say once a month) or any combinations thereof. For example, the medicament may be administered daily for the first week and twice weekly for 4 subsequent weeks. Or, in another example, the medicament can be administered to a subject bi-weekly for the first 2 weeks of treatment and then monthly for further 3 months.

As used herein, the term “treatment” refers to any and all uses which remedy a disease state or symptoms, prevent the establishment of disease, or otherwise prevent, hinder, retard, or reverse the progression of disease or other undesirable symptoms in any way whatsoever.

The term “treat” or “treating” as used herein is intended to refer to providing an pharmaceutically effective amount of a peptide or a respective pharmaceutical composition or medicament thereof, sufficient to act prophylactically to prevent the development of a weakened and/or unhealthy state; and/or providing a subject with a sufficient amount of the complex or pharmaceutical composition or medicament thereof so as to alleviate or eliminate a disease state and/or the symptoms of a disease state, and a weakened and/or unhealthy state.

The fusion protein as described herein and above can be formulated into compositions, for example pharmaceutical compositions, suitable for administration. Where applicable, a peptide or a protein may be administered with a pharmaceutically acceptable carrier. A “carrier” can include any pharmaceutically acceptable carrier as long as the carrier can is compatible with other ingredients of the formulation and not injurious to the patient. Accordingly, pharmaceutical compositions for use may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Thus, in one example, the present disclosure describes a pharmaceutical composition comprising, but not limited to, a peptide as described herein, an isolated nucleic acid molecule for expressing said peptide, or a vector for amplifying said isolated nucleic acid molecule as referred to herein. In another example, the present disclosure describes an isolated nucleic acid molecule encoding a peptide as described herein. In yet another example, the present disclosure describes a vector comprising an isolated nucleic acid molecule as described herein. In one example, the pharmaceutical composition comprises a peptide as described herein. In yet another example, the pharmaceutical composition further comprises one or more pharmaceutically acceptable excipients, vehicles or carriers. Therefore, in one example, the peptide as disclosed herein may further comprise a compound selected from, but not limited to, a pharmaceutically acceptable carrier, a liposomal carrier, an excipient, an adjuvant or combinations thereof.

The composition, shape, and type of dosage forms of the peptide as disclosed herein will typically vary depending on the intended use. For example, a dosage form used in the acute treatment of a disease or a related disease may contain larger amounts of one or more of the active compound it comprises than a dosage form used in the chronic treatment of the same disease. Similarly, a parenteral dosage form may contain smaller amounts of one or more of the active compound it comprises than an oral dosage form used to treat the same disease or disorder. These and other ways in which specific dosage forms encompassed by this invention will vary from one another will be readily apparent to those skilled in the art. Examples of dosage forms include, but are not limited to: tablets; caplets; capsules, such as soft elastic gelatine capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms particularly suitable for parenteral administration to a patient; and sterile solids (e.g., crystalline or amorphous solids) that can be reconstituted to provide liquid dosage forms suitable for parenteral administration to a patient. Thus, in one example, the peptide as disclosed herein is provided in a form selected from, but not limited to, tablets, caplets, capsules, hard capsules, soft capsules, soft elastic gelatine capsules, hard gelatine capsules, cachets, troches, lozenges, dispersions, suppositories, ointments, cataplasms, poultices, pastes, powders, dressings, creams, plasters, solutions, patches, aerosols, nasal sprays, inhalers, gels, suspensions, aqueous liquid suspensions, non-aqueous liquid suspensions, oil-in-water emulsions, a water-in-oil liquid emulsions, solutions, sterile solids, crystalline solids, amorphous solids, solids for reconstitution or combinations thereof.

The composition can be administered to the subject in any suitable way, including: parenterally, topically, orally, by inhalation spray, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

The pharmaceutically acceptable carrier can comprise any suitable diluent, adjuvant, excipient, buffer, stabiliser, isotonicising agent, preservative or anti-oxidant. It will be appreciated that the pharmaceutically acceptable carrier should be non-toxic and should not interfere with the efficacy of the fusion protein. The precise nature of the carrier or any other additive to the composition will depend on the route of administration and the type of treatment required. Pharmaceutical compositions can be produced, for instance, by means of conventional mixing, dissolving, granulating, dragee-making, levigating, emulsifying, encapsulating, entrapping or lyophilizing processes.

Sterile injectable forms of the composition can be aqueous or oleaginous suspension. Such forms will be known to those of skill in the art. For intravenous, cutaneous or subcutaneous injection, or injection at a site where treatment is desired, the composition may be in the form of a parenterally acceptable aqueous solution which has suitable pH, isotonicity and stability.

Orally acceptable dosage forms of the composition include, but are not limited to, capsules, tablets, pills, powders, liposomes, granules, spheres, dragees, liquids, gels, syrups, slurries, suspensions and the like. Suitable oral forms will be known to those of skill in the art. A tablet can include a solid carrier such as gelatine or an adjuvant or an inert diluent. Liquid pharmaceutical compositions generally include a liquid carrier such as water, petroleum, animal or vegetable oils, a mineral oil or a synthetic oil. Physiological saline solution, or glycols such as ethylene glycol, propylene glycol or polyethylene glycol may be included. Such compositions and preparations will generally contain at least 0.1 wt % of the chimeric fusion protein and, in one example, up to about 25 wt %, depending on its solubility in the given carrier.

The composition can be administered topically, especially when the target of treatment includes areas or organs readily accessible by topical application, including cancers of the eye, the skin, or the lower intestinal tract. The composition may be applied in the form of a solution, suspension, emulsion, ointment, cream, lotion, paste, gel, foam, or aerosol. Suitable topical forms will be known to those of skill in the art.

The composition can include a delivery vehicle for delivering the compound to a particular organ, tissue or type of cancer, and/or for ensuring that the compound is able to be, for instance, absorbed through the skin or ingested through the gut without loss of biological efficacy. Delivery vehicles can comprise, for example, lipids, polymers, liposomes, emulsions, antibodies and/or proteins. Liposomes are particularly preferred for delivering the compound through the skin.

The composition can be delivered using a sustained-release system, such as semipermeable matrices of solid hydrophobic polymers containing the compound. Various sustained-release materials are available and well known by those skilled in the art. Sustained-release capsules may, depending on their chemical nature, release the compound for about 1 to 20 weeks.

A subject can be administered the composition together with one or more other actives to achieve an optimal prophylactic or therapeutic effect. The actives may be, for example, alkylating agents, angiogenesis inhibitors, anti-androgens, anti-estrogens, anti-metabolites, apoptosis agents, aromatase inhibitors, cell cycle controlling agents, cell stressor, cytotoxics, cytoprotectant, hormonals, immunotherapy agents, kinase inhibitors, monoclonal antibodies, platinum agents, a respiratory inhibitor, retinoid, signal transduction inhibitors, taxanes and topoisomerase inhibitors.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

As used herein, the following terms have the meanings ascribed to them unless specified otherwise.

As used in the specification and claims, the singular forms “a”, “an” and “the” include plural references unless the context clearly dictates otherwise. For example, the term “a cell” includes a plurality of cells, including mixtures thereof.

As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

A “fragment” is a truncated form of a native biologically active protein that retains at least a portion of the therapeutic and/or biological activity.

A “chimeric” protein contains at least one fusion polypeptide comprising regions in a different position in the sequence than that which occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

“Conjugated”, “linked,” “fused,” and “fusion” are used interchangeably herein. These terms refer to the joining together of two or more chemical elements or components, by whatever means including chemical conjugation or recombinant means.

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide.

“Recombinant” means the product of various combinations of in vitro cloning, restriction and/or ligation steps, and other procedures that result in a construct that can potentially be expressed in a host cell.

As used herein, the term “PE” refers to Pseudomonas Exotoxin A (‘PE’), which is a toxic virulence factor of the bacterium Pseudomonas aeruginosa. PE is expressed as a nascent protein with a length of 638 amino acids, but a highly hydrophobic leader peptide of 25 amino acids at its N-terminus is cleaved during secretion. PE comprises different functional and structural domains. Following the leader peptide, PE has a receptor binding Domain Ia (amino acids 1 to 252) which is composed of antiparallel B-sheets, and Domain II (amino acids 253 to 364) with six consecutive a-helices, which enables it to translocate across cell membranes, for example, from the endoplasmatic reticulum to the cytosol. Following Domain II is Domain Ib (amino acids 365 to 404) and Domain III (amino acids 405 to 613). The last four residues (amino acids 400 to 404) of Domain Ib together with Domain III form the catalytic subunit of the toxin with ADP-ribosyltransferase activity.

### DESCRIPTION OF EMBODIMENTS

Preferred features, embodiments and variations of the invention may be discerned from this section, which provides sufficient information for those skilled in the art to perform the invention. This section is not to be regarded as limiting the scope of any preceding section in any way.

### Materials and Methods

### Gene Synthesis and Cloning

The sequences of the tPE (SEQ ID NO: 10), tPE-H1 (SEQ ID NO: 14), and tPE-H1neg (SEQ ID NO: 13) were synthesized and cloned in a pET100/D-TOPO vector using gene synthesis (Thermo Fisher Scientific, Carlsbad, Calif.).

### Cell Line Culture and Stable Transduction

All cell lines come from ATCC. Lentiviruses were generated according to the manufacturer's instructions (Invitrogen). WT A431 cells were transduced with Cignal Lenti Myc Reporter (Qiagen) expressing Luciferase upon E-Box promoter and CMV-Renilla Control CLS-RCL expressing Renilla upon CMV promoter. Single clone was selected after colony isolation and Luciferase/Renilla expression level tested. All cell lines (A431 and HepG2) were maintained in high-glucose Dulbecco's modified Eagle's medium supplemented with 10% foetal calf serum at 37° C. in a 10% CO₂ incubator. All experiments were performed on cells passaged fewer than 10 times after thawing.

### Luciferase and Renilla Activity Reading

40,000 A431-Ebox-luc-CMV-Ren cells were seeded in a 96-well plate (Falcon) 48 hours. Luminescence is detected using the Promega Dual-Glo luciferase assay system, according to the manufacturer's protocol and read using Tecan Infinite M200 microplate reader using 100 ms integration time.

### Bacterial Expression

Purified plasmids previously described were introduced into the E. coli strain Epicurian BL21 (Stratagene, USA). Cultures were grown at 37° C. until the A600 reached 0.5 before the induction of protein expression by addition of isopropyl-b Dthiogalactopyranoside (0.1 mM) in the LB culture medium. After 2 hours of induction at 37° C., bacteria were recovered by centrifugation for 30 minutes at 3000 g at 4° C.

Purification of tPE, tPE-H1 and tPE-H1 neg

Bacteria expressing tPE, tPE-H1 or tPE-H1neg were resuspended in 3 mL of lysis buffer (Tris 50 mM pH 8, NaCl 170 mM, imidazole 20 mM, urea 6 M, NP40 0.5% v/v) for a pellet corresponding to 60 mL bacterial culture.

The solution was homogenized and sonicated. Insoluble material was discarded by centrifugation for 30 minutes at 16,000 g at 4° C. Soluble tPE, tPE-H1 or tPE-H1neg were purified on Ni-NTA affinity chromatography resin (Qiagen, USA). Resin was washed with 10 volumes of lysis buffer incubated with a bacterial extract containing tPE, tPE-H1 or tPE-H1neg for overnight on orbital wheel at 4° C. Resin was then washed twice with 10 volumes of washing buffer (Tris 50 mMpH 8, NaCl 170 mM, imidazole 40 mM, urea 6 M, NP40 0.5% v/v). tPE, tPE-H1 or tPE-H1neg were eluted twice with 1 volume of elution buffer (Tris 50 mM pH 8, NaCl 170 mM, imidazole 1 M, urea 6 M, NP40 0.5% v/v).

Eluate was injected in Slide-A-lyzer Dialysis Cassette 20,000 MWCO (Thermo Scientific) in 3 baths of 100 volumes of PBS for 8 to 16 hours. Solubility is tested by spinning down the dialysate 30 minutes at 15,000 g and comparison with the amount of purified protein in the supernatant with the pellet fraction resuspended in the same volume.

tPE, tPE-H1, tPE-H1neg Quantification and Quality Control

Purified proteins quantification was done by Bradford by comparing the optical density at 595 nm (0D595) nm with a BSA standard.

Purity control was made by running samples denaturated in laemmli blue and heated at 95° C. for 5 minutes on SDS-PAGE follow by instablue staining.

tPE-H1 activity was tested by incubation at concentrations of 10 to 100 nM using an A431 E-BoxLuc/CMVRen dose response model. Requirements are that tPE-H1 EC₅₀ must be in 25 nM range at 6 hours incubation at 37° C. with absence of effect on renilla. tPE and tPE H1neg must have non-significant effect on luciferase and Renilla.

tPE, tPE-H1 incubation at 200 nM for 1 hour was tested on MG63 (human bone osteoscarcoma) cells followed by cell lysis to show the cellular uptake and cell fractionation to show their presence in the nuclear fraction.

tPE, tPE-H1 incubation at 500 nM for 1 hour was tested on MG63 followed by immunofluorescence with antibody targeting PE to show their presence in Nuclear Associated Endosomes (NAE).

tPE, tPE-H1 incubation at 50 nM for 6 hours was tested on a A431 E-BoxLuc/CMVRen model after 72 hours RNAi knock down of sec61 B and SUN2. Requirement is that a rescue on the luciferase in case of the knock down must be observed.

### RNAi Knock Down

Knock down experiments were performed in 384-well plates (384 black clear; Greiner). Reverse transfection was performed with 25 nM siRNA with 7.5 μl Opti-MEM (GIBCO, Invitrogen), 10% HiPerFect (QIAGEN) per well. After 20 minutes of complex formation, 5000 A431 EBOX-Luciferase/CMV-Renilla were added to each well and incubated at 37° C., 10% CO2 for 72 hours. Sec61 B siRNA sequence: GCAAGUACACUCGUUCGUA. SUN2 siRNA sequence: CCUAUGGGCUGCAGACAUU.

Cell Fractionation after tPE-H1 Treatment

Cells were seeded at the desired densities in six-well dishes (Thermo Fisher Scientific) and incubated at 37° C., 10% CO₂ for 16 hours. Cells were treated for 1 hour with mock (untreated cells) or tPE-H1 200 nM before fractionation with Cell Surface Protein Isolation Kit (#89881, Thermo Fisher Scientific) according to the manufacturer's protocol. In addition, cytosolic fraction is centrifuge at 15 000 g for 15 minutes to remove membrane contaminants. Samples are denaturated in Laemmli blue 2X and heat-denaturated for 5 minutes at 95° C. Samples are loaded on SDS-PAGE followed by western blot.

Co-Immunoprecipitation after tPE or tPE-H1 Treatment

A431 cells seeded at the desired densities in six-well dishes (Thermo Fisher Scientific) and incubated at 37° C., 10% CO₂ for 16 hours. Cells were treated for 1 hour with mock, tPE or tPE-H1 200 nM before lysis in RIPA buffer for 20 minutes at 4° C. and centrifugation for 20 minutes at 15 000 g at 4° C. Soluble fractions were added with c-myc antibody (#32, Abcam) on orbital wheel for 4 hours at 4° C. Protein A Sepharose (GE Healthcare) was added and incubated overnight on orbital wheel at 4° C. After three washes, proteins were eluted in one volume of Laemmli blue 2X and heat-denaturated for 5 minutes at 95° C. Samples were run on SDS-PAGE before western blot. Membrane are blocked in milk and incubated with anti Max (#199489, Abcam) and anti c-Myc (#32072, Abcam).

Cell Proliferation after tPE-H1 Treatment

Cells were seeded at 10 to 20% confluency. 24 hours later, the cells were incubated with different doses of tPE-H1 at 37° C., 10% CO₂ for 14 days. Live imaging was performed using IncuCyte ZOOM® Live-Cell Analysis System. Bright field Images were taken every 6 hours. Images were analysed using IncuCyte ZOOM® Live-Cell Analysis software.

DRAQ7 Assay after tPE-H1 Treatment

25000 HepG2 cells were seeded per well in a 96-well plate. After incubation at 37° C. for 24 hours, DRAQ7 dye was prepared with 100 nM tPE-H1 at 500 times dilution

Cell mortality was tracked by acquiring nine fields of brightfield and far-red images at 20× magnification every 4 hours using the Operetta machine (Perkin-Elmer) for 3 to 5 days. The machine kept the plate incubated at 37° C. with 8% CO₂.

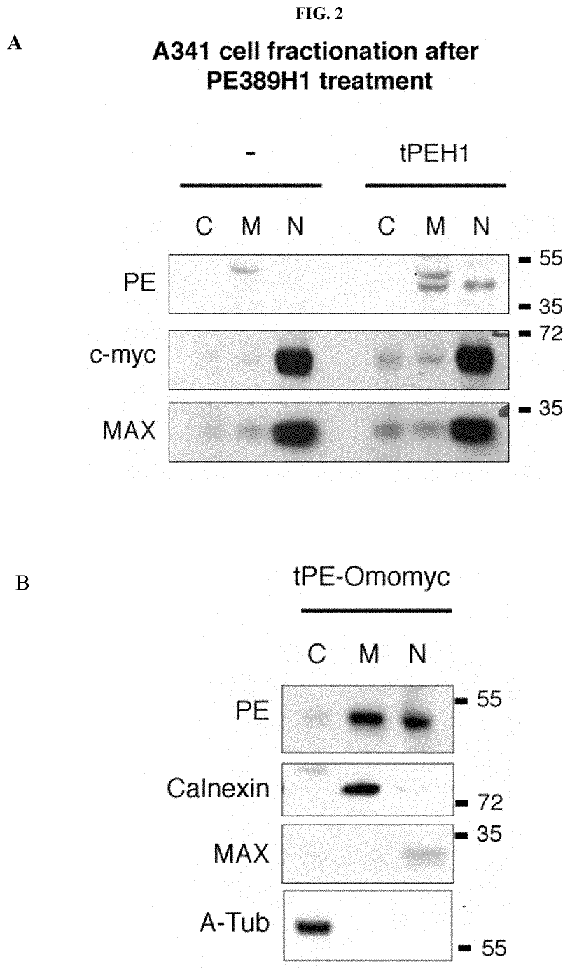

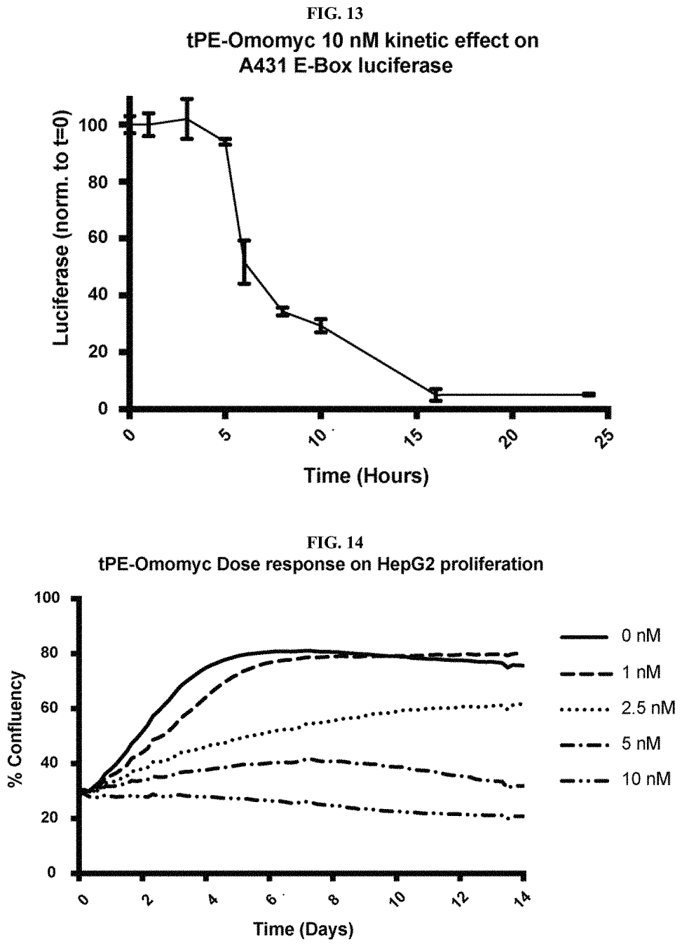

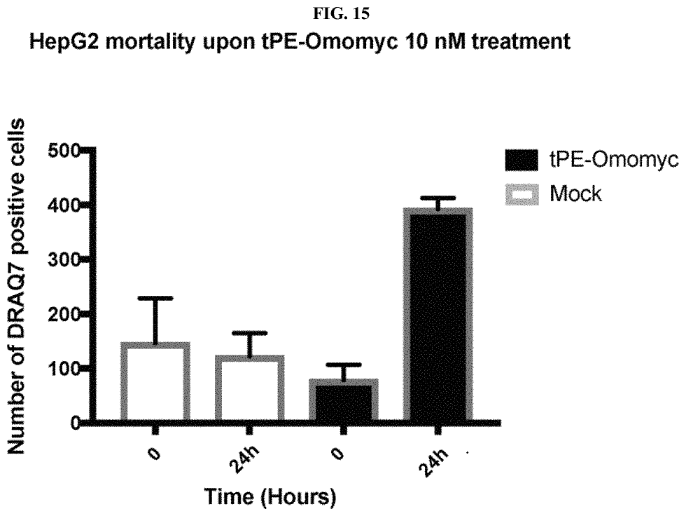

It is of note that the experiments outlined above were also performed using the tPE-Omomcy fusion protein, results of which are shown in FIG. 2B, and FIGS. 12 to 15. The concentration of the tPE-Omomyc fusion protein is as stated on the x-axis in FIGS. 12 and 14; 200 nM for FIG. 2B; and 10 nM for FIGS. 13 and 15.

RT-PCRQ after tPE-H1 Treatment

400000 A431 cells were seeded in each well in a 6 well plate (Falcon) 24 hours before incubation with 100 nM tPE-H1 for 16 hours at 37° C. Total RNA was isolated using the RNeasy Mini Kit (Qiagen, Ref. no 74106) as described by manufacturer's protocol.

10 μg total RNA was reversed transcribed using SuperScript III First-Strand Synthesis System for RT-PCR (Invitrogen, Cat. no. 18080051) as described by manufacturer's protocol.

The relative expression of mRNAs of c-Myc regulated genes identified in RT² Profiler™ PCR Array Human MYC Targets (Qiagen, Cat. no. PAHS-177Z) was determined by real-time quantitative RT-PCR. Briefly, a PCR component mix containing cDNA, RT² SYBR Green Mastermix (Qiagen, Cat. no. 330523) and RNase-free water is prepared. 25 μl of the PCR reaction was added to each well in one array plate and subjected to a qPCR program using the ABI 7500. PCR cycling conditions comprised of an initial denaturation step at 95° C. for 10 minutes followed by an amplification program for 40 cycles of 15 seconds at 95° C., and 60 seconds at 60° C. with fluorescence acquisition at the end of each extension. The relative expression of each gene between treated and untreated samples is calculated using the comparative ΔΔCT method, using the mock treated sample as calibrator and housekeeping gene HPRT as internal control.

### RESULTS AND DISCUSSION

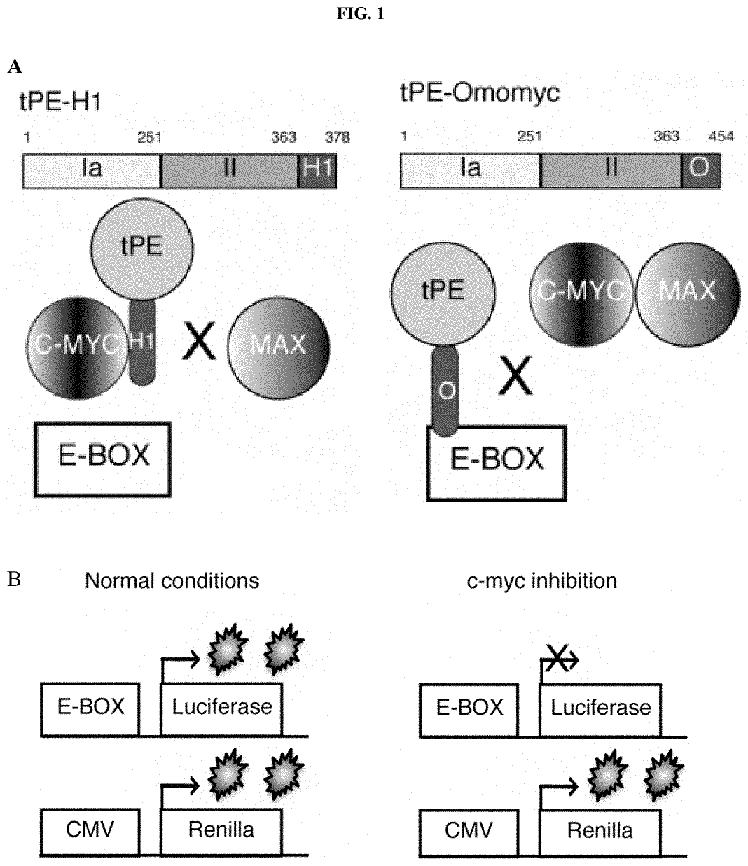

The inventors developed a genetically modified version of Pseudomonas Aeroginosa Exotoxin A (‘tPE’) that was able to penetrate mammalian cells and reach their nucleus. The inventors fused the tPE to a c-Myc inhibitor, namely, H1 peptide (‘H1’), creating ‘tPE-H1’, Omomyc (resulting in tPE-Omomyc), as shown in FIG. 1.

The inventors showed that tPE-H1 efficiently negatively regulated genes controlled by c-Myc and slowed down cell proliferation. tPE-H1 was at least 1000 times more efficient than H1 fused to cell penetrating peptides such as TAT, CAD or int (antenapedia) (a method previously proposed to deliver peptides into cells).

To test the activity of tPE-H1, the inventors established a stable cell line expressing luciferase upon the control of c-Myc promoter E-Box. In normal conditions, c-Myc interacts with the transcription factor Max, binds to the E-Box element and promotes transcription. H1 disrupted the interaction between c-Myc and Max, which led to a decrease in luciferase activity. As a control, cells expressed Renilla upon CMV promoter control. See FIG. 1B.

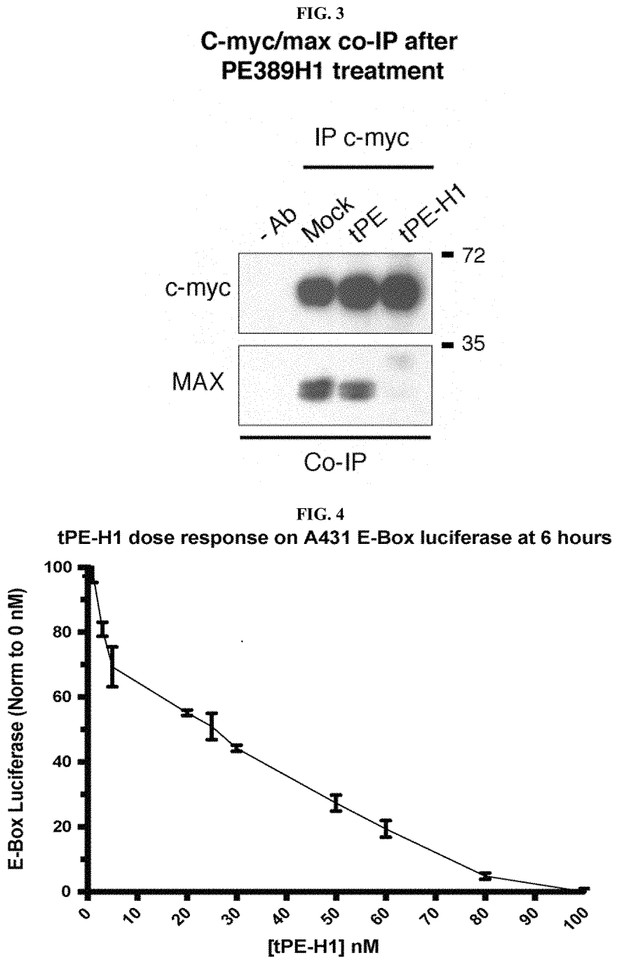

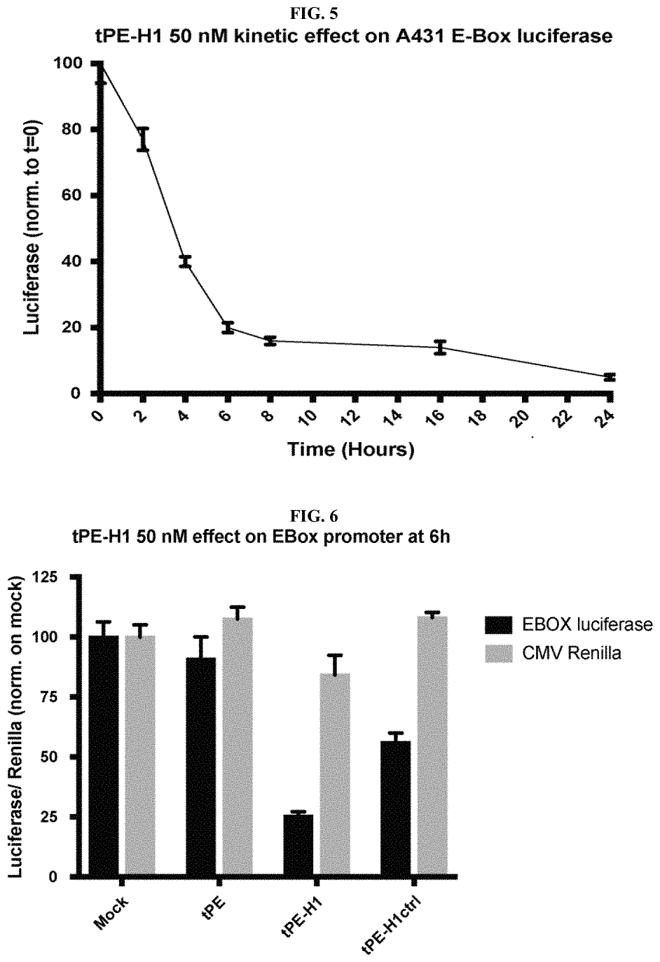

tPE-H1 was found to reach the nucleus in less than 1 hour after incubation, as seen in FIG. 2. tPE-H1 disrupted the c-Myc/Max interaction as evidenced by loss of co-IP of Max with c-Myc (see FIG. 3). Dose response showed a decrease of c-Myc-dependent luciferase activity with an EC50=25 nM (see FIG. 4) at 6 hours. Maximal inhibition occurred after 8 hours of incubation and was stable over more than 24 hours (see FIG. 5). Luciferase decrease was specifically due to H1 peptide transported to the nucleus by tPE. tPE itself and a mutated version of H1 (NELKRAFAALRDQI; SEQ ID NO: 5 had no or a negligible effect on luciferase (see FIG. 6).

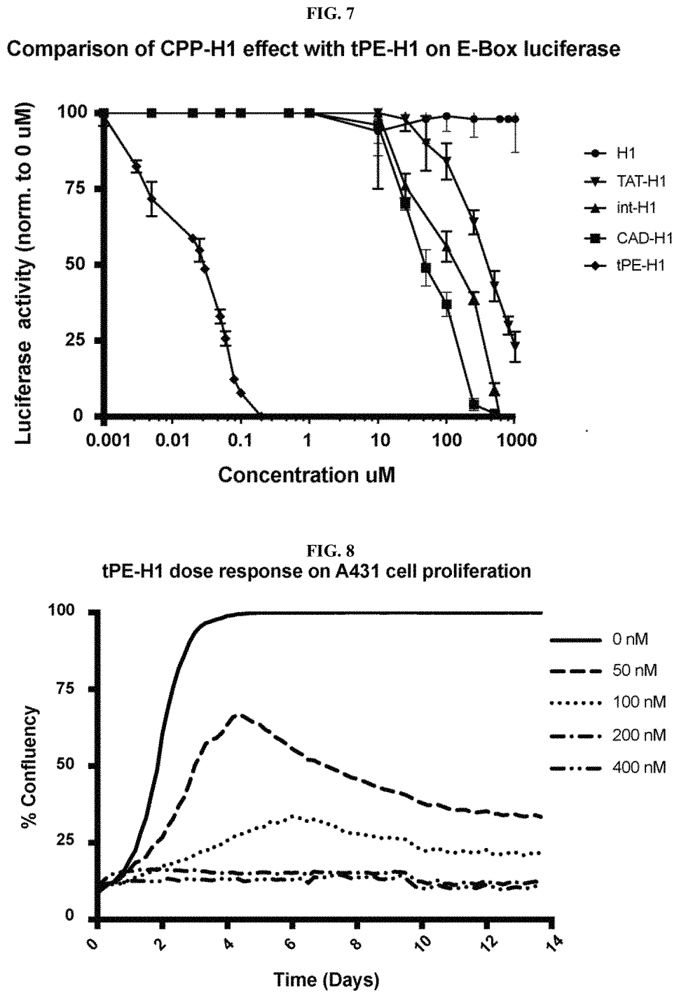

The most commonly used cell targeting peptides (CPP) fused with H1 showed an EC₅₀ of 75 μM, 200 μM and 500 μM for respectively Cadherin, Antenapedia and TAT compared to tPE-H1 on A431 expressing luciferase controlled by E-Box after 6 hours of incubation (see FIG. 7) (CAD, LLIILLRRRIRKQAHAHSK, SEQ ID NO: 2; Int, RQIKIWFQNRRMKWKK, SEQ ID NO: 3; TAT, GRKKRRQRRRPPQ, SEQ ID NO: 4).

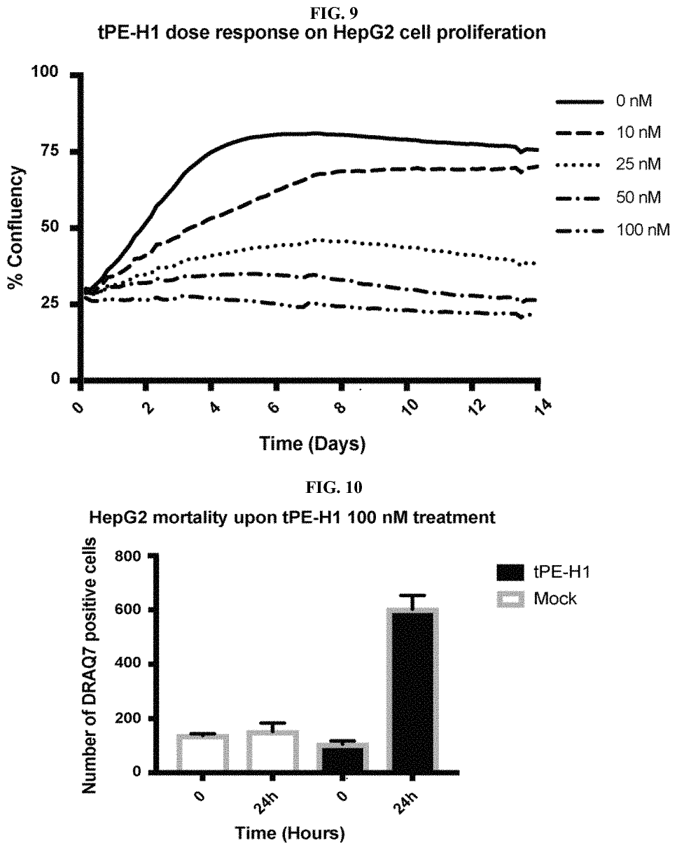

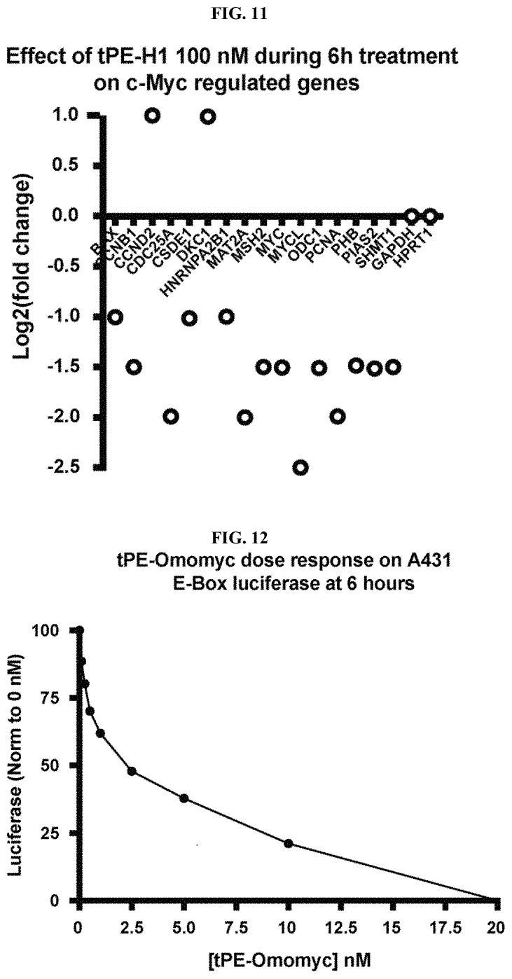

A431 cells treated with tPE-H1 showed a dose dependent decrease in cell proliferation and a total absence of growth above 200 nM (see FIG. 8). However, comparison with different cell lines showed a higher sensitivity of hepatocarcinoma cells HepG2 with an absence of cell growth above 50 nM (see FIG. 9) and cells death after 24 hours at 100 nM showing a potential high tPE-H1 efficiency in hepatocarcinoma treatment (see FIG. 10). A431 cells showed a decrease of numerous upregulated genes under tPE-H1 treatment and an increase of numerous down regulated genes, but not control gene in this experimental condition, showing an on-target effect (see FIG. 11).

Omomyc is a myc dominant negative peptide able to bind E-Box promoter and prevent c-myc binding. tPE was coupled to the Omomyc peptide, creating tPE-Omomyc, a schematic of which is shown in FIG. 1. It is shown that tPE-Omomyc is located in nuclear fraction after 1 hour incubation (FIG. 2B).

To test the activity of tPE-Omomyc, a dose response on A431 E-Box-Luciferase/CMV-Renilla was performed and showed a decrease of Myc-dependent luciferase activity with an EC₅₀=5 nM (FIG. 12) at 6 hours. Maximal inhibition occurs after 16 hours of intoxication and is stable over more than 24 hours (FIG. 13).

Hepatocarcinoma cells HepG2 that show a high sensitivity to tPE-H1 were treated with different doses of tPE-Omomyc and show a decrease in cell proliferation and a total absence of growth above 10 nM (FIG. 14). DRAQ7, a marker that specifically stains dead cells, showed a significant increase after 10 nM of tPE-Omomyc treatment after 24 hours (FIG. 15). In summary, the results obtained show that tPE-Omomyc has an even higher potency than tPE-H1. Specifically, tPE-Omomyc shows an EC₅₀ value of 5 nM in luciferase assays; and 10 nM of tPE-Omomyc is able to stop the hepatocarcinoma HepG2 cell growth and induce cell death. All the cell lines tested were around 10 times more sensitive to tPE-H1 than tPE-Omomyc.

It is known in the art that the peptide Omomyc shows the ability to cross the plasma membrane passively, despite its intrinsic physico-chemical properties. Thus, Omomyc appears to be self-sufficient in its ability to target c-Myc without further peptides or localisation sequences to help with cellular internalisation.

In the present application, the fusion of H1 or Omomyc to tPE allows their delivery to the nucleus by following the NAE pathway and results in an unexpectedly low EC₅₀ value of 25 nM and 5 nM, respectively. This is shown in the results of c-myc inhibition experiments with luciferase as utilised herein. Moreover, 100 nM of tPE-H1 and 10 nM tPE-Omomyc are sufficient to block HepG2 cell proliferation and induce cell death/apoptosis which is surprisingly low compared to previously described effect of Omomyc. Different promoter affinities account for specificity in MYC-dependent gene regulation. It is further shown in the art that Omomyc EC50 varies drastically between various assays, and a skilled person would appreciated that it is the hard to compare EC₅₀ values when data is not generated using the same assay.

While it has been shown in the art that A549 cells which were treated with an Omomyc concentration of 10 μM showed apoptosis in cells, this is in contrast to the data shown in the present application in FIG. 15 (showing the cell mortality of cells treated with 10 nM of tPE-Omomyc), wherein it is shown that cells are sensitive to 10 nM of tPE-Omomyc. This also applies in different cell lines tested by the inventors (for example, but not limited to, HeLa, A431, MG63, MDA-MD231, and HCT116; data not shown), all of which were shown to be sensitive to tPE-Omomyc in a concentration of about 10 nM.

The mechanism for either H1 or Omomyc fused to tPE are the same. Omomyc has the ability to diffuse through the plasma membrane (thereby being capable of being found in the cytosol). H1 itself does not have this ability. Thus, in the present application, the inventors are forcing these peptides to take an alternative uptake route to bring these peptides into the nucleus.

It is known that the PE wildtype (wt) protein follows the retrograde traffic to reach the cytosol. It is known that payloads or cargo can be fused to PE domains Ia and II (tPE in the present application), thus enabling cytosolic delivery. It is known that the PE wildtype (wt) protein follows the NAE pathway to be translocated into the nucleus. However, no data has shown tPE to be transported to the nucleus. Also, previously, no data has shown that tPE when fused with a peptide or a protein results in the fusion peptide being transported into the nucleus.

With regard to the Omomyc fusion peptide as disclosed herein, one would have thought that adding the Omomyc peptide (10 kDa) to the tPE peptide (which is 40 kDa in size) would create a fusion protein of 50 kDa, which is known in the art to be too big to passively cross the nuclear pore complex. Therefore, one would not expect such a large protein of 50 kDa to be delivered to the nucleus, unless a nuclear localisation signal is added. In the same manner, tPE-H1 is about 42 kDa and is not able to passively cross the nuclear pore complex.

Moreover, a skilled person would have assumed that the kinetic for tPE to reach the cytosol via the retrograde trafficking would be around to 4 to 6 hours, and would require a μM concentration range in order to efficiently inhibit c-Myc in the nucleus.

A skilled person would have further assumed that the risk of tPE steric effect is high on both H1 and Omomyc, which in turn is expected to affect the affinity of any tPE fusion protein to the E-Box.

It was found by the inventors that Omomyc coupled with tPE reaches the nucleus within 1 hour of application. In the luciferase assay described herein, tPE-Omomyc results in an EC₅₀ value of 5 nM and tPE-H1 EC₅₀ value of 25 nM. It is further shown that 10 nM of tPE-Omomyc and 100 nM tPE-H1 are sufficient to completely inhibit hepatocarcinoma cell proliferation and induce cells death/apoptosis.

In view of what is known in the art, examples of which are outline above, the inventors have provided data which is unexpected in light of what is shown and described in the prior art with regard to the use of H1 and Omomyc.

The peptides, for example the H1 peptides as disclosed herein, showed activity in vivo when fused to tPE. Such examples include MEEAFDLWNECAKACVLDLKDGVRSSRMSVDPAIADTNGQGVLHYSMVLEGGNDALKLAIDN ALSITSDGLTIRLEGGVEPNKPVRYSYTRQARGSWSLNWLVPIGHEKPSNIKVFIHELNAGNQLS HMSPIYTIEMGDELLAKLARDATFFVRAHESNEMQPTLAISHAGVSVVMAQAQPRREKRWSEW ASGKVLCLLDPLDGVYNYLAQQRCNLDDTWEGKIYRVLAGNPAKHDLDIKPTVISHRLHFPEGG SLAALTAHQACHLPLETFTRHRQPRGWEQLEQCGYPVQRLVALYLAARLSWNQVDQVIRNAL ASPGSGGDLGEAIREQPEQARLALTLAAAESERFVRQGTGNDEAGAASNELKRAFAALRDQI (SEQ ID NO: 14), and others, as shown for example in SEQ ID NO: 15.

In summary, both tPE-H1 and tPE-Omomyc were found to exhibit surprising efficacy against c-Myc. A H1 peptide conjugated to cell-targeting peptides could be expected, by a person skilled in the art, to function as well as tPE-H1. However, it has been shown when H1 was fused to two types of cell targeting peptides (Bac and SynB1), the reported effective concentrations were at ˜40 μM for C6 cell line cell killing. The present inventors tested a fusion peptide with the cell targeting peptides Int-H1, CAD-H1 and Tat-H1. The inventors observed some effects of Tat-H1 in the 500 μM range, CAD-H1 in the 75 μM range and int-H1 in the 200 μM range (see FIG. 7). By contrast, tPE-H1 was active at 25 nM. In fact, the inventors observed >90% decrease in Myc reporter activity after incubation with tPE-H1 50 nM and <75% decrease reporter activity with Tat-H1 at 1 mM, CAD-H1 at 150 μM and Int-H1 at 400 μM.

In sum, tPE-H1 was between 1000 and 10000 fold more efficient than H1 fused to cell targeting peptides, a result that could not have been predicted with the current state of knowledge. By way of an example, previously obtained data with int-H1 had been obtained using a commercial int-H1 construct. However, difficulties were observed when attempting to reproducing results using the commercial int-H1 construct as the expected effects were not observed. It was then decided to synthetize all the CPP to repeat the experiments and in order to ensure comparable synthesis conditions. Using this approach, the inventors obtained the results described herein.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In the present specification and claims, the word ‘comprising’ and its derivatives including ‘comprises’ and ‘comprise’ include each of the stated integers but does not exclude the inclusion of one or more further integers.

The reference to any prior art in this specification is not, and should not be taken as an acknowledgement or any form of suggestion that the prior art forms part of the common general knowledge.

1. A chimeric fusion protein comprising a c-Myc inhibitor fused to genetically modified Pseudomonas Aeroginosa Exotoxin A (‘tPE’), said fusion protein being capable of penetrating a nucleus of a cell and inhibiting c-Myc activity within the nucleus.

2. A pharmaceutical composition comprising a chimeric fusion protein comprising a c-Myc inhibitor fused to genetically modified Pseudomonas Aeroginosa Exotoxin A (‘tPE’), said fusion protein being capable of penetrating a nucleus of a cell and inhibiting c-Myc activity within the nucleus.

3.-5. (canceled)

6. A method of preventing or treating a c-Myc-dependent cancer in a subject, said method comprising the step of administering to the subject a c-Myc inhibitor fused to genetically modified Pseudomonas Aeroginosa Exotoxin A (‘tPE’), said fusion protein being capable of penetrating a nucleus of a cell of the subject and inhibiting c-Myc activity within the nucleus.

7.-8. (canceled)

9. The fusion protein of claim 1, wherein the c-Myc inhibitor: is capable of disrupting c-Myc dependent pathways in c-Myc dependent cancers; interferes with specific c-Myc DNA binding; or, blocks c-Myc/Max dimerization.

10. The fusion protein of claim 1, wherein the c-Myc inhibitor is fused to a C-terminus of the tPE.

11. The fusion protein of claim 1, wherein the tPE comprises PE Domain Ia or a biologically active fragment thereof.

12. The fusion protein of claim 1, wherein the tPE comprises PE Domain II or a biologically active fragment thereof.

13. The fusion protein of claim 1, wherein the tPE comprises PE Domain Ia and PE Domain II.

14. The fusion protein of claim 13 wherein the c-Myc inhibitor is fused to the C-terminus of Domain II of tPE.

15. The fusion protein of claim 1, wherein the c-Myc inhibitor is a H1 peptide derived from the helix 1 (H1) carboxylic region of c-Myc.

16. The fusion protein of claim 15, wherein the H1 peptide has the sequence NELKRAFAALRDQI (SEQ ID. NO: 5).

17. The fusion protein of claim 1, wherein the chimeric protein comprises an N-terminal polyhistidine tag.

18. The fusion protein of claim 1, wherein the fusion protein has the sequence shown in SEQ ID NO.: 14 or SEQ ID NO: 15.

19. The fusion protein of claim 1, wherein the cell is a cancerous cell of the cervix, colon, breast, lung, or stomach.

20. The method of claim 6, wherein the subject is a human.

21. The fusion protein of claim 11, wherein the tPE comprises PE Domain Ia or a biologically active fragment thereof, having the sequence shown in SEQ ID NO: 10 or SEQ ID NO: 11.

22. The fusion protein of claim 12, wherein the tPE comprises PE Domain II or a biologically active fragment thereof, having the sequence shown in SEQ ID NO: 10 or SEQ ID NO: 12.

23. The fusion protein of claim 13, wherein the tPE comprises PE Domain Ia and PE Domain II, having the sequence shown in SEQ ID. NO: 10.


SEQUENCE LISTING (17 sequences)

SEQ ID NO: 1 - protein, 14 aa, Artificial sequence
  H1-control (H1-neg; original unmutated H1 sequence)
NELKRSFFALRDQI

SEQ ID NO: 2 - protein, 19 aa, Artificial sequence
  Cadherin (CAD)
LLIILLRRRIRKQAHAHSK

SEQ ID NO: 3 - protein, 16 aa, Artificial sequence
  Antenapedia (Int)
RQIKIWFQNRRMKWKK

SEQ ID NO: 4 - protein, 13 aa, Artificial sequence
  TAT
GRKKRRQRRRPPQ

SEQ ID NO: 5 - protein, 14 aa, Artificial sequence
  Mutated H1 (S6A, F8A; also referred to as tPE-H1)
NELKRAFAALRDQI

SEQ ID NO: 6 - protein, 90 aa, Artificial sequence
  Omomyc
TEENVKRRTHNVLERQRRNELKRSFFALRDQIPELENNEKAPKVVILKKATAYILSVQAETQKLISEIDLLRKQNEQLKHKLEQLRNSCA

SEQ ID NO: 7 - protein, 30 aa, Artificial sequence
  Int-H1
RQIKIWFQNRRMKWKKNELKRAFAALRDQI

SEQ ID NO: 8 - protein, 33 aa, Artificial sequence
  CAD-H1
LLIILLRRRIRKQAHAHSKNELKRAFAALRDQI

SEQ ID NO: 9 - protein, 27 aa, Artificial sequence
  Tat-H1
GRKKRRQRRRPPQNELKRAFAALRDQI

SEQ ID NO: 10 - protein, 364 aa, Artificial sequence
  tPE (comprising both PE domains Ia and II)
MEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAAS

SEQ ID NO: 11 - protein, 251 aa, Artificial sequence
  tPE comprising only the PE Domain Ia
EEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPE

SEQ ID NO: 12 - protein, 112 aa, Artificial sequence
  tPE comprising only the PE Domain II
GGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAAS

SEQ ID NO: 13 - protein, 378 aa, Artificial sequence
  tPE-H1-control (tPE-H1-neg; H1 portion is highlighted)
  MISC_FEATURE (365)..(378): H1 portion
MEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASNELKRSFFALRDQI

SEQ ID NO: 14 - protein, 378 aa, Artificial sequence
  tPE-H1 (mutated H1 sequence highlighted)
  MISC_FEATURE (365)..(378): mutated H1 sequence
MEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASNELKRAFAALRDQI

SEQ ID NO: 15 - protein, 454 aa, Artificial sequence
  tPE- Omomyc (Omomyc sequence highlighted)
  MISC_FEATURE (365)..(454): Omomyc sequence
MEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASTEENVKRRTHNVLERQRRNELKRSFFALRDQIPELENNEKAPKVVILKKATAYILSVQAETQKLISEIDLLRKQNEQLKHKLEQLRNSCA

SEQ ID NO: 16 - RNA, 19 nt, Artificial sequence
  Sec61B siRNA sequence
gcaaguacacucguucgua

SEQ ID NO: 17 - RNA, 19 nt, Artificial sequence
  SUN2 siRNA sequence
ccuaugggcugcagacauu